(19) United States
(12) Patent Application Publication
(10) Pub. No.: US 2009/0234194 A1
(43) Pub. Date: Sep. 17, 2009

(54) TONGUE DEPRESSOR

(76) Inventor: Samir Bhatt, Wellesley, MA (US)

Correspondence Address: EDWARDS ANGELL PALMER & DODGE LLP, P.O. BOX 55874, BOSTON, MA, 02205, US

(21) Appl. No.: 12/161,615

(22) PCT Filed: Jan. 25, 2007
(86) PCT No.: PCT/US07/02311

§ 371 Date: Nov. 19, 2008

Related U.S. Application Data
(60) Provisional application No. 60/763,007, filed on Jan. 28, 2006.

(51) Int. Cl. A61B 1/32

(52) U.S. Cl. 600/240

(57) ABSTRACT

Embodiments of the present invention feature a device and method for depressing the tongue during surgical procedures. The device features a first planar element, a second planar element and at least one bridge element. Each planar element has a top surface, a bottom surface, an inside edge, an outside edge, a front end, a back end, a length and a width. The length from the front end to the back end corresponds to the distance between the front exterior of the oral cavity to an area of the tonsils. The bottom surface is for placing on the top of the tongue with the front edge into the oral cavity and the back edge toward the exterior of the oral cavity. The top surface creates a chamber with the palate of the patient for viewing and performing surgical procedures. The outside edge of each planar element is for positioning buccally, with the inside edges facing each other spaced apart a distance sized for a laryngeal mask airway device or endotracheal tube, to create a channel space. The bridge element is affixed to each planar element spanning the channel space, allowing at least fifty percent of the area of the channel space to remain open.

### RELATED APPLICATION

This application claims the benefit of U.S. Provisional Application Ser. No. 60/763,007 filed Jan. 28, 2006, the entire contents of which are incorporated herein by reference.

### FIELD OF THE INVENTION

Embodiments of the present invention relate to medical devices and procedures for controlling the position of tongue and/or ventilation tubes during surgery.

### BACKGROUND OF THE INVENTION

Tonsillectomy and adenoidectomy are widely performed operations. Current standard technique uses a “mouth gag”. This mouth gag consists of two parts: a metal tongue depressor and a ring shaped device that holds the tongue depressor in position. The tongue depressor portion of the mouth gag is designed to depress the tongue during the operation and afford the surgeon hands free exposure to the oral cavity and oropharynx. Because the patients undergoing this operation are under general anesthesia, there is also an endotracheal tube providing ventilation. The endotracheal tube needs to be held out of the way during surgery.

There are currently several designs of tongue depressors in use, including the widely used Davis and Doughty varieties. The Davis tonsil mouth gag and tongue depressor is L-shaped with a vertical portion and a tongue portion. The vertical portion does not touch the patient and attaches to the rest of the mouth gag device. The tongue portion is designed to depress the tongue. The tongue portion is made of stainless steel and has a midline groove running its length. The groove depresses the endotracheal tube onto the middle of the tongue during the operation thereby holding it out of the surgical field.

In the Doughty variety, a midline slit in the tongue portion depresses the endotracheal tube instead of a groove. Neither the Davis nor the Doughty varieties fits well with laryngeal mask airways (LMAs) being used for general anesthesia during tonsillectomy procedures, causing problems with surgical access and safe patient ventilation.

The present mouth gags are associated with several problems. During insertion, these devices rub against LMA breathing tubes in a way that coils, kinks or displaces the LMA, causing difficult or inadequate patient ventilation. During removal, these mouth gags trap the breathing tubes in their grooves making accidental removal a dangerous possibility. The groove in the Davis mouth gag bulges into the surgical field at times, obscuring the surgeon's view and access to portions of the throat during surgery, especially when linear instruments, such as micro-debraders are used.

### SUMMARY OF THE INVENTION

Aspects of the present invention feature devices and methods for depressing the tongue during surgical procedures. One aspect of the present invention provides a device for depressing the tongue comprising a first planar element, second planar element and at least one bridge element. The first planar element and second planar element each have a top surface, a bottom surface, an inside edge, an outside edge, a front end, a back end, a length and a width. The length from the front end to the back end corresponds to the distance between the front exterior of the oral cavity to an area of the tonsils. The bottom surface is for placing on the top of the tongue with the front end into the oral cavity and the back end toward the exterior of the oral cavity. The top surface creates a chamber with the palate of the patient for viewing and performing surgical procedures. The outside edges of the first planar element and the second planar element are for positioning buccally and the inside edges of the first and second planar elements are facing each other are spaced apart a distance sized for at least one of the ventilation means selected from the group of laryngeal mask airway devices and endotracheal tubes to create a channel space. Each one bridge element is affixed to the first planar element and the second planar element spanning the channel space, and allowing at least fifty percent of the area of the channel space open.

The open channel space allows the surgeon to view the ventilation means during use of the device. During insertion and removal, the surgeon may view the ventilation means to insure that the ventilation means is properly positioned. The surgeon can also manually move or position the ventilation means to accommodate the needs of the surgical procedure. The one or more bridge elements allow the surgeon to have substantial view of the surgical field.

As used herein, the term “corresponds” means approximately equal to. That is, by way of example, the length of the first and second planar elements is approximately equal to the distance from the front of the oral cavity to the area around the tonsils. The area around the tonsils includes the area to the front of the tonsils and the area behind the tonsils.

Preferably, the device has at least two bridge elements. And, preferably, each bridge element has a length and a width. Each bridge element is affixed to each top surface of the first and second planar elements. And, preferably, each bridge element is an arch extending toward the palate to allow ventilation means to be retained between the tongue, the inside edges of the first and second planar elements, and the bridge element. Thus, the bridge element forms an arc of a partial or semi- or half circle corresponding generally to the dimensions of the ventilation means.

A preferred width is one to ten millimeters, and, more preferably, two to four millimeters. The length corresponds to the width of the ventilation means used. Thus, the arc of a partial, semi- or half circle has a diameter of four to twenty-five millimeters, and, even more preferably, ten to twenty millimeters. The inside edges of the first planar element and the second planar element are thus spaced in a range of approximately four to twenty-five millimeters and, preferably, ten to twenty millimeters.

Preferably, where there are two bridge elements, the forward bridge element, furthest into the oral cavity, is approximately fifteen to thirty-five millimeters from the front end, and, more preferably, about twenty to about thirty millimeters from the front end. The back bridge element, closest to the lips, is about ten to about thirty millimeters from the back end, and, more preferably, about fifteen to about twenty-five millimeters from the back end.

Preferably, each first planar element and each second planar element have a straight portion and a curved portion. The curved portion is toward the front end and corresponds to the curvature of the tongue moving downward into the larynx. The straight portion is toward the back edge of each planar element. Preferably, the curved portion creates an arc from a distance in the range of approximately about ten to about twenty-five millimeters from the front end. And, the front edge is depressed a distance in the range of about five to about fifteen millimeters from a plane formed by the straight portion.

Preferably, the device has a handle affixed to one or more of the first planar element and the second planar element. The handle extends from the back end of at least one of the first and second planar elements to allow the surgeon to firmly grasp the device for positioning in the oral cavity. A preferred handle is an extension of one of the first planar element and the second planar element. The handle extends at an angle from about 45 to about 135 degrees from at least one of the bottom surfaces to place the handle away from the surgical field. Preferably, the handle rests on a surface place on the patient's chest when the first planar element and second planar element are positioned against the tongue in the oral cavity.

Preferably, the device has a hook for hanging on a surgical apparatus. In a preferred embodiment, the hook is part of the handle.

A further aspect of the present invention provides a method for depressing the tongue. The method comprises the step of providing a device having a first planar element, second planar element and at least one bridge element. The first planar element and second planar element each have a top surface, a bottom surface, an inside edge, an outside edge, a front end, a back end, a length and a width. The length is the distance from the front end to the back end and corresponds to the distance between the front exterior of the oral cavity and an area of the tonsils. The bottom surface is for placing on the top of the tongue with the front edge into the oral cavity and the back edge toward the exterior of the oral cavity. The top surface is for creating a chamber with the palate of the patient for viewing and performing surgical procedures. The outside edge of the first planar element and the second planar element is for positioning buccally, with the inside edges facing each other. The inside edges are spaced apart a distance sized for at least one of the ventilation means selected from the group of laryngeal mask airway devices and endotracheal tubes to create a channel space. The bridge element is affixed to the first planar element and the second planar element spanning the channel space. In one embodiment, the bridging element allows at least about fifty percent of the area of the channel space to be open. The method further comprises the step of fitting the device in the oral cavity to depress the tongue.

The fitting of the device is facilitated with a handle extending from at least one of the first and second planar elements. In one embodiment, the device may be stored conveniently with a hanging means to be available to a surgeon for next use.

These and other features and advantages of the present invention will be apparent to those skilled in the art upon viewing the accompanying drawings and reading the detailed description of the invention that follow.

### BRIEF DESCRIPTION OF THE DRAWINGS

FIG. 1 is a side, slightly elevated, view of a device embodying features of the present invention;

FIG. 2 is a front view of a device embodying features of the present invention; and,

FIG. 3 is a top view of a device embodying features of the present invention.

### DETAILED DESCRIPTION OF THE INVENTION

Embodiments of the present invention will be described in detail as a device and method for depressing the tongue by surgeons in performing surgical operations. Those skilled in the art will recognize that embodiments of the present invention have utility in other fields as well. For example, without limitation, embodiments of the present invention have application for veterinary procedures. However, it is readily understood that the dimensions of the device will need to be adjusted for anatomical differences and that such adjustments are well within the skill of those skilled in the art. Further, it is understood that the dimensions set forth in the present disclosure are directed to normal values for adults. The dimensions can be altered for application for infants, toddlers and children.

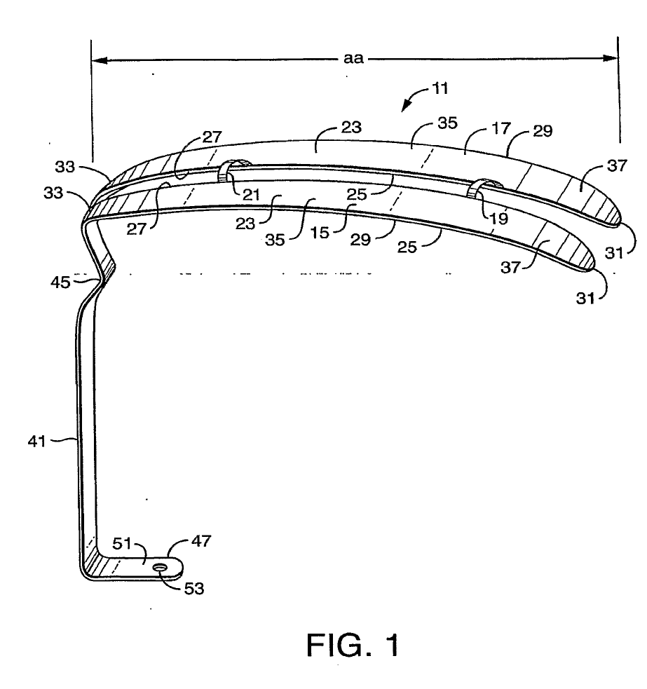

Turning now to FIG. 1, a device for depressing the tongue embodying features of the present invention is depicted, generally designated by numeral 11. Device 11 has a first planar element 15, second planar element 17 and at least one bridge element of which two, designated by numerals 19 and 21, are depicted. The device 11 is a surgical instrument and is preferably made of surgical grade materials such as stainless steel. However, in the event the device is intended for a single use, materials such as plastics well known to the surgical arts may be readily used.

The first planar element 15 and second planar element 17 each has a top surface 23, a bottom surface 25, an inside edge 27, an outside edge 29, a front end 31, a back end 33, a length and a width. As used herein, in this context, the term “length” refers to the distance from the back end 33 to the front end 31 as denoted by line “aa”. This distance, from the front end 31 to the back end 33 corresponds to the distance between the front exterior of the oral cavity to an area of the tonsils. The area around the tonsils includes the area to the front of the tonsils and the area behind the tonsils. A surgeon may choose a device 11 in which the first planar element 15 and second planar element 17 are shorter or longer, extending in front of or behind the tonsils, depending on the surgeon's needs.

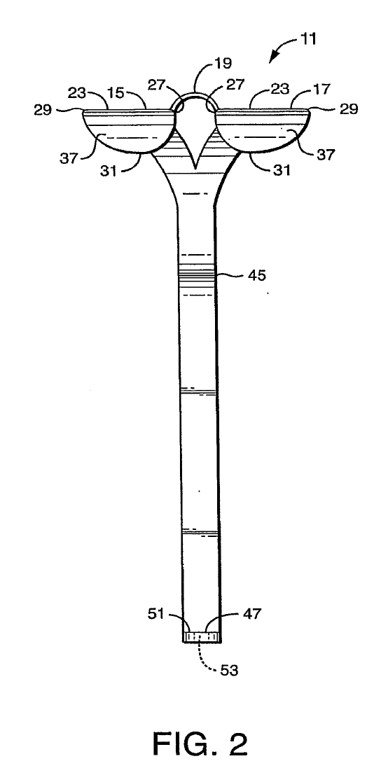

Each bottom surface 25 is for placing on the top of the tongue with the front end 31 into the oral cavity and the back end 33 toward the exterior of the oral cavity. Referring now to FIG. 2, the top surface 23 creates a chamber with the palate (not shown) of the patient for viewing and performing surgical procedures.

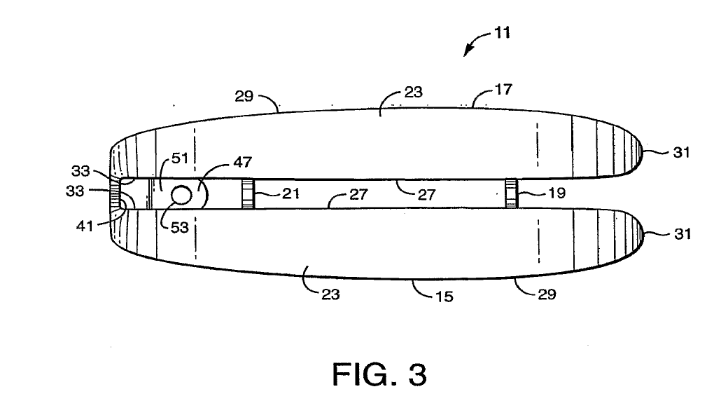

Referring now to FIGS. 2 and 3, each outside edge 29 of the first planar element 15 and the second planar element 17 is for positioning buccally, preferably, within the area of teeth. The inside edges 27 of the first planar element 15 and the second planar element 17 face each other, spaced apart a distance sized for at least one of the ventilation means selected by the surgeon on anesthesiologist. Well known ventilation means comprise laryngeal mask airway devices (not shown) and endotracheal tubes (not shown). Thus, the inside edges 27 are spaced in a range of approximately four to twenty-five millimeters, and, preferably, ten to twenty millimeters, to create a channel space.

Returning to FIG. 1, each bridge element 19 and 21 is affixed to the first planar element 15 and the second planar element 17 spanning the channel space. Turning to FIG. 3, each bridge element 19 and 21 is affixed to each top surface 23 of the first and second planar elements 15 and 17. As depicted, each bridge element 19 and 21 is an arch extending toward the palate to allow ventilation means (not shown) to be retained between the tongue, the inside edges 27 of the first and second planar elements 15 and 17, and each bridge element 19 and 21. Thus, each bridge element 19 and 21 forms an arc of a partial, semi- or half circle, preferably with a diameter of about four to about twenty-five millimeters, and, more preferably, about ten to about twenty millimeters. This diameter corresponds generally to the dimensions of the ventilation means.

Each bridge element 19 and 21 has a length and a width. The length is dictated by the diameter of the arch and the width of the channel space between the inside edges 27 of the first planar element 15 and second planar element 17. A preferred width is one to ten millimeters, and, more preferably, three to six millimeters. The bridge elements 19 and 21 are constructed and arranged to allow at least fifty percent and, more preferably, at least eighty percent, of the area of the channel space to be open.

The open channel space serves to allow the surgeon to view the ventilation means as well as to enlarge the viewable surgical field. During insertion and removal the surgeon may view the ventilation means to insure that the ventilation means is properly positioned. And, the surgeon can manually move or position the ventilation means to accommodate the needs of the surgical procedure. The two bridge elements 19 and 21 slide easily over the ventilation means. The device 11 is easily removed without unintentionally withdrawing the ventilation means.

Turning now to FIGS. 1 and 2, the first planar element 15 and the second planar element 17 have a straight portion 35 and a curved portion 37. The curved portion 37, shown with hatch-lines, is toward the front end 31 and corresponds to the curvature of the tongue moving downward into the larynx. The straight portion 35 is toward the back edge of each planar element. Preferably, the curved portion 37 creates an arc beginning its curve a distance in the range of approximately ten to twenty-five mm from the front end 31. And, the front end is depressed a distance in the range of about five to about fifteen millimeters from a plane formed by the straight portion 35. As used herein in the context of the straight portion 35, the term “straight” is a relative term. The term encompasses a slight degree of curvature, which corresponds, with the shape of the end of tongue in the oral cavity.

Again referring to FIGS. 1 and 2, the device 11 has a handle 41 affixed to one or both of the first planar element 15 and the second planar element 17. As depicted, the handle 41 extends from the back end 33 of both the first planar element 15 and second planar element 17 to allow the surgeon to firmly grasp the device 11 for the positioning in the oral cavity.

As best seen in FIG. 1, the handle 41 has an indent 45, which may be preferred by some surgeons. Above the indent 45, the surgeon can place the index finger and grip the handle 41 below the indent with the remaining fingers to improve his or her grip. The handle 41 extends at an angle from about 45 to about 135 degrees from the bottom surface 25 to place the handle away from the surgical field. Preferably, the handle rests on a surface placed on the patient's chest when the first planar element 15 and second planar element 17 are positioned against the tongue in the oral cavity.

Returning now to FIGS. 1 and 2, the device 11 has hanging means in the form of a hook 47 for hanging the device 11 on a surgical cart or table (not shown). The hook 47 extends from the handle 45 a distance to clear the patients chest when the device is in place but long enough to allow the hook 47 to rest and balance the device 11 as the device 11 is turned upside down with the flat surface 51 resting on the top of a surgical table with the device 11 suspended below. The end of the hook 47 may be flared to allow the hook to be received in a corresponding notch in the table or surgical cart, as best seen in FIG. 2. Other hanging means comprise hole 53 which allow the device 11 to be received in a pin (not shown), or the hook 47 may extend up toward the first planar surface 15 allowing the device 11 to be placed on a bar.

The present invention also provides a method for depressing the tongue using the device 11. The method comprises the step of providing a device 11 having a first planar element 15, second planar element 17 and at least one bridge element 19 and 21. Gripping the handle 45, the surgeon fits or places the bottom surface 25 on the top of the tongue with the front end 31 into the oral cavity and the back end 33 toward the exterior of the oral cavity. The top surface 23 creates a chamber with the palate of the patient for viewing and performing surgical procedures. The outside edge 29 of the first planar element 15 and the second planar element 17 are positioned buccally.

The inside edges 25 are spaced apart a distance sized for at least one of the ventilation means selected from the group of laryngeal mask airway devices and endotracheal tubes to create a channel space. The bridge elements 19 and 21 affixed to the first planar element 15 and the second planar element 17, span the channel space. The bridging element allows at least about fifty percent of the area of the channel space to be open.

Upon completion of the operation or procedure, the device 11 is removed, sterilized and stored conveniently with the hook means 47 to be available to a surgeon for the next procedure.

Thus, the present invention has been described with respect to the preferred embodiment with the understanding that such embodiments can be altered and modified without departing from the teaching herein. Therefore, the present invention should not be limited to the precise details and description herein but should encompass the subject matter of the claims that follow and their equivalents.

1. A device for depressing the tongue comprising a first planar element, second planar element and at least one bridge element, said first planar element and second planar element each having a top surface, a bottom surface, an inside edge, an outside edge, a front end, a back end, a length and a width, said length from said front end to said back end corresponding to the distance between the front exterior of the oral cavity to an area of the tonsils, said bottom surface for placing on the top of the tongue with said front edge into the oral cavity and said back edge toward the exterior of the oral cavity and said top surface for creating a chamber with the palate of the patient for viewing and performing surgical procedures, said outside edge of first planar element and said second planar element for positioning buccally, said inside edges facing each other spaced apart a distance sized for at least one of the ventilation means selected from the group of laryngeal mask airway devices and endotracheal tubes to create a channel space, said bridge element affixed to said first planar element and said second planar element spanning said channel space, said bridging element allowing at least fifty percent of the area of said channel space open.

2. The device of claim 1 comprising at least two bridge elements

3. The device of claim 1 wherein said at least one bridge element has a length and a width and said width is one to ten millimeters.

4. The device of claim 1 wherein said at least one bridge element is affixed to each top surface of said first planar element and said second planar element.

5. The device of claim 4 wherein said bridge element has an arch said arch extending toward said palate to allow ventilation means to be retained between the tongue, said inside edges of said first planar surface and said second planar surface and said bridge element.

6. The device of claim 5 wherein said inside edges of said first planar element and said second planar element are spaced in a range of approximately four to twenty-five millimeters.

7. The device of claim 1 wherein said first planar element and said second planar element has a straight portion and a curved portion said curved portion toward said front end and corresponding to the curvature of the tongue moving downward into the larynx and said straight portion toward said back edge.

8. The device of claim 7 wherein said curved portion creates an arc from a distance in the range of approximately ten to twenty-five millimeters from the front end.

9. The device of claim 8 wherein said front edge is depressed a distance in the range of five to fifteen millimeters from a plane formed by said straight portions.

10. The device of claim 1 further comprising a handle affixed to one or more of said first planar element and said second planar element at said back end.

11. The device of claim 10 wherein said handle extends at an angle from about 45 to about 135 degrees from at least one of said bottom surfaces selected from said first planar element and said second planar element.

12. The device of claim 12 wherein said handle rests on a surface on the patient's chest when said first planar element and second planar element are positioned against said tongue in the oral cavity.

13. The device of claim 1 further comprising a hook for hanging on a surgical apparatus.

14. A method for depressing the tongue comprising the steps of providing a device having a first planar element, second planar element and at least one bridge element, said first planar
element and second planar element each having a top surface, a bottom surface, an inside edge, an outside edge, a front end, a back end, a length and a width, said length from said front end to said back end corresponding to the distance between the front exterior of the oral cavity to an area of the tonsils, said bottom surface for placing on the top of the tongue with said front edge into the oral cavity and said back edge toward the exterior of the oral cavity and said top surface for creating an chamber with the palate of the patient for viewing and performing surgical procedures, said outside edge of first planar element and said second planar element for positioning buccally, said inside edges facing each other spaced apart a distance sized for at least one of the ventilation means selected from the group of laryngeal mask airway devices and endotracheal tubes to create a channel space, said bridge element affixed to said first planar element and said second planar element spanning said channel space, said bridging element allowing at least fifty percent of the area of said channel space open and fitting said device in the oral cavity to depress the tongue.

15. The method of claim 14 wherein said device has at least two bridge elements.

16. The method of claim 14 wherein said at least one bridge element has a length and a width and said width is one to ten millimeters.

17. The method of claim 14 wherein said at least one bridge element is affixed to each top surface of said first planar element and said second planar element.

18. The method of claim 17 wherein said bridge element has an arch said arch extending toward said palate to allow ventilation means to be retained between the tongue, said inside edges of said first planar surface and said second planar surface and said bridge element.

19. The method of claim 18 wherein said inside edges of said first planar element and said second planar element are spaced in a range of approximately four to twenty-five millimeters.

20. The method of claim 14 wherein said first planar element and said second planar element has a straight portion and a curved portion said curved portion toward said front end and corresponding to the curvature of the tongue moving downward into the larynx and said straight portion toward said back edge.

21. The method of claim 20 wherein said curved portion creates an arc from a distance in the range of approximately ten to twenty-five millimeters from the front end.

22. The method of claim 21 wherein said front edge is depressed a distance in the range of five to fifteen millimeters from a plane formed by said straight portions.

23. The method of claim 14 further comprising a handle affixed to one or more of said first planar element and said second planar element at said back end and said method comprises the step of grasping the handle to position the first planar element and said second planar element.

24. The method of claim 23 wherein said handle extends at an angle from about 45 to about 135 degrees from at least one of said bottom surfaces selected from said first planar element and said second planar element.

25. The method of claim 24 said handle is positioned to rest on a surface on the patient's chest when said first planar element and second planar element are positioned against said tongue in the oral cavity.

26. The method of claim 14 further comprising a hook for hanging on a surgical apparatus and said method comprises storing the device by hanging by said hook.
